(19) United States
(12) Patent Application Publication
(10) Pub. No.: US 2026/0325289 A1
(43) Pub. Date: Oct. 22, 2026

(54) TRANSCATHETER PROSTHETIC VALVE WITH MULTI-PART FRAME SUBCOMPONENT TRANSVERSE DEFORMATION RESISTANCE

(71) Applicant: Edwards Lifesciences Corporation, Irvine, CA (US)

(72) Inventors: Logan R. Hagaman, Flagstaff, AZ (US); Cody L. Hartman, Flagstaff, AZ (US)

(21) Appl. No.: 19/458,078

(22) Filed: Jan. 23, 2026

Related U.S. Application Data
(63) Continuation of application No. 17/632,120, filed on Jul. 31, 2020, now abandoned.
(60) Provisional application No. 62/881,428, filed on Aug. 1, 2019.

(51) Int. Cl. A61F 2/24

(52) U.S. Cl. CPC A61F 2/2418; A61F 2220/0025; A61F 2250/0063

(57) ABSTRACT

A prosthetic valve includes an anchor frame, a leaflet frame, and leaflets within the leaflet frame. An interstage tube couples the anchor frame to the leaflet frame. The valve has an un-nested configuration in which the leaflet frame and the anchor frame are axially separated from one another by a gap, with the interstage tube bridging the gap. The valve also has a nested configuration in which the leaflet frame is secured within the anchor frame. The valve is configured to transition from the un-nested configuration to the nested configuration by the interstage tube everting and becoming sandwiched between the anchor frame and the leaflet frame to provide an interference fit between the anchor frame and the leaflet frame. Other embodiments are also described.

### CROSS-REFERENCE TO RELATED APPLICATION

This application is a Continuation of U.S. patent application Ser. No. 17/632,120, filed Feb. 1, 2022, which published as US 2022/0273426, and which is the national phase of PCT Application No. PCT/US2020/044603, internationally filed on Jul. 31, 2020, which published as WO 2021/022211, and which claims the benefit of Provisional US Patent Application No. 62/881,428, filed Aug. 1, 2019.

Each of the above applications is incorporated herein by reference in its entirety for all purposes.

### FIELD

The present disclosure relates generally to prosthetic valves and more specifically to flexible leaflet-type prosthetic valve devices, systems and methods that are adapted for endovascular delivery.

### BACKGROUND

Bioprosthetic valves have been developed that attempt to mimic the function and performance of a native valve. Bioprosthetic valves may be formed from synthetic materials, natural tissue such as biological tissue, or a combination of synthetic materials and natural tissue.

Though many conventional designs require delivery to a target region within a patient's anatomy via open-heart surgical techniques, alternative approaches such as transcatheter techniques offer a number of advantages. Among other examples, a transcatheter prosthetic valve that is delivered endovascularly via a catheter can help to minimize patient trauma as compared with an open-heart, surgical procedure. Open-heart surgery involves extensive trauma to the patient, with attendant morbidity and extended recovery. On the other hand, a valve delivered to the recipient site via a catheter avoids the trauma of open-heart surgery and may be performed on patients too ill or feeble to survive the open-heart surgery.

However, challenges exist with accessing treatment regions within the anatomy, properly positioning the bioprosthesis for deployment, and ultimately, prosthesis efficacy, among others.

### SUMMARY

Various inventive concepts of the examples addressed herein relate to configurations for achieving enhanced transverse deformation resistance in prosthetic valves utilizing overlapping, multi-frame subcomponent configurations, including associated systems and methods of use and manufacture thereof.

According to one example (“Example 1”), a prosthetic valve transitionable between a delivery configuration and a deployed configuration in-situ includes an anchor frame subcomponent adapted to anchor to tissue, the anchor frame subcomponent having a total length between an inflow end and an outflow end of the anchor frame subcomponent, an inner diameter, and a first deployed transverse deformation resistance, the anchor frame subcomponent including an interface region defining a shoulder feature. In some implementations, the interface region extends less than the total length of the anchor frame subcomponent, though in others the interface region extends the total length of the anchor frame subcomponent. The prosthetic valve also includes a leaflet frame subcomponent including a leaflet assembly coupled to the leaflet frame subcomponent along a leaflet coupling region, the leaflet frame subcomponent having a total length between an inflow end and an outflow end of the leaflet frame subcomponent, an outer diameter, and a second deployed transverse deformation resistance, the leaflet frame subcomponent having an interface region extending at least along the leaflet coupling region, the interface region defining a complementary shoulder feature to the shoulder feature of the anchor frame subcomponent, the anchor frame subcomponent and the leaflet frame subcomponent being adapted to be transitioned from an un-nested configuration in which the leaflet frame subcomponent and the anchor frame subcomponent are separated from one another to a nested configuration in which the anchor frame subcomponent and the leaflet frame subcomponent engage along a combined interface corresponding to the interface region of the anchor frame subcomponent and the interface region of the leaflet frame subcomponent via an interference fit with the shoulder feature and the complementary shoulder features engaged and such that the prosthetic valve has a combined interface transverse deformation resistance along the interior and interface regions that exceeds each of the first and second deployed transverse deformation resistances of the anchor and leaflet frame subcomponents, respectively.

According to another example (“Example 2”), further to the device of Example 1, the shoulder feature and the complementary shoulder feature include complementary tapered profiles.

According to another example (“Example 3”), further to the device of any preceding Examples, in the nested configuration an outflow end of the prosthetic valve has a transverse deformation resistance that is less than the combined interface transverse deformation resistance.

According to another example (“Example 4”), further to the device of and preceding Example, the prosthetic valve includes an inflow region, an outflow region, and an intermediate region between the inflow and outflow ends, the intermediate region having the combined interface transverse deformation resistance and the inflow and outflow regions each having a transverse deformation resistance that is less than the combined interface transverse deformation resistance.

According to another example (“Example 5”), further to the device of any preceding Example, the prosthetic valve includes an inflow region having a varying diameter to define an inflow taper, an outflow region having a varying diameter to define an outflow tapered region, and an intermediate region between the inflow and outflow ends, the intermediate region having a constant diameter to define an un-tapered intermediate region.

According to another example (“Example 6”), further to the device of any preceding Example, the nested configuration of the prosthetic valve includes the inner diameter of the anchor frame subcomponent and the outer diameter of the leaflet frame subcomponent being the same along the leaflet coupling region of the leaflet frame subcomponent.

According to another example (“Example 7”), further to the device of any preceding Example, wherein the combined interface has a length that extends from an inflow side of the leaflet assembly to an outflow side of the leaflet assembly.

According to another example (“Example 8”), further to the device of any preceding Example, the anchor frame subcomponent is configured to retain a compacted, undeployed profile until expanded to an expanded, deployed profile by an expansion force.

According to another example (“Example 9”), further to the device of any preceding Example, the anchor frame subcomponent is configured to be balloon expandable.

According to another example (“Example 10”), further to the device of any preceding Example, the leaflet frame subcomponent and/or the anchor frame subcomponent is configured to be self-expanding.

According to another example (“Example 11”), further to the device of any preceding Example, the leaflet frame subcomponent includes a leaflet frame formed of shape memory material.

According to another example (“Example 12”), further to the device of any preceding Example, the device further includes an interstage subcomponent coupling the outflow end of the anchor frame subcomponent to the inflow end of the leaflet frame subcomponent.

According to another example (“Example 13”), further to the device of Example 12, the nested configuration includes the interstage subcomponent defining an everted configuration.

According to another example (“Example 14”), further to the device of Examples 12 or 13, the interstage subcomponent is operable to permit blood flow therethrough when the leaflet frame subcomponent is not nested in the anchor frame subcomponent, and is operable to restrict flow therethrough when the leaflet frame subcomponent is nested within the anchor frame subcomponent.

According to another example (“Example 15”), further to the device of any one of Examples 12 to 14, the interstage subcomponent includes one or more reinforcement elements operable to maintain the leaflet frame subcomponent and the anchor frame subcomponent in the nested configuration.

According to another example (“Example 16”), further to the device of any one of Examples 12 to 15, the interstage subcomponent includes a continuous sinuous reinforcement element formed separate and distinct from any frame subcomponent element of the anchor frame subcomponent and the leaflet frame subcomponent.

According to another example (“Example 17”), further to the device of any one of Example 12 to 16, the interstage subcomponent includes a cover material.

According to another example (“Example 18”), further to the device of any preceding Example, the anchor frame subcomponent includes a plurality of tissue anchoring elements operable to engage tissue.

According to another example (“Example 19”), further to the device of any preceding Example, the leaflet assembly further includes a plurality of leaflets, and optionally wherein the leaflets includes a composite material including a matrix material and a filler material, and optionally wherein the matrix material includes a porous synthetic fluoropolymer membrane defining pores and the filter material includes a fluoropolymer material, such as a TFE-PMVE copolymer.

According to another example (“Example 20”), a medical system includes a delivery catheter, and a prosthetic valve according to any preceding Example mounted to the delivery catheter with the anchor frame subcomponent and the leaflet frame subcomponent in the un-nested configuration with the anchor frame subcomponent and the leaflet frame subcomponent each being configured in a compacted, delivery profile.

According to another example (“Example 21”), a method of treating a native valve orifice of a patient's anatomy with a prosthetic valve includes advancing, with the medical system of Example 20, the anchor frame subcomponent and the leaflet frame subcomponent in the compacted, delivery configuration to a treatment site, wherein the leaflet frame subcomponent and the anchor frame subcomponent are longitudinally offset from one another such that the inflow end of the leaflet frame subcomponent are in an un-nested configuration, deploying the anchor frame subcomponent, and nesting the leaflet frame subcomponent within the anchor frame subcomponent by changing a relative position between the leaflet frame subcomponent and the anchor frame subcomponent and deploying the leaflet frame subcomponent.

According to another example (“Example 22”), further to the method of Example 21, the leaflet frame subcomponent is nested with the outer fame such that the outflow end of the leaflet frame subcomponent extends longitudinally beyond the outflow end of the anchor frame subcomponent and the inflow end of the leaflet frame subcomponent extends longitudinally beyond the inflow end of the anchor frame subcomponent.

According to another example (“Example 23”), further to the method of Examples 21 or 22, flow is maintained through the prosthetic valve at least during the time following deploying the anchor frame subcomponent and prior to deploying the leaflet frame subcomponent.

The foregoing Examples are just that, and should not be read to limit or otherwise narrow the scope of any of the inventive concepts otherwise provided. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic, sectional view a prosthetic valve implanted at a treatment site (e.g., a native valve orifice), according to some embodiments;

FIG. 2 is a sectional, schematic view of the prosthetic valve in an un-nested configuration, according to some embodiments;

FIG. 3 is a sectional, schematic view of the prosthetic valve in a nested configuration, according to some embodiments;

FIG. 4 is a representation of the prosthetic valve in an un-nested configuration that shows additional, optional features (e.g., frame configurations), according to some embodiments;

FIG. 5 is a representation of the prosthetic valve in a nested configuration that shows additional, optional features (e.g., frame configurations) similar to FIG. 4, according to some embodiments;

FIG. 6 shows an optional reinforcement element and FIG. 7 shows another example of optional reinforcement elements of the prosthetic valve, according to some embodiments;

FIG. 8 shows the prosthetic valve mounted on a delivery device and FIG. 9 shows the prosthetic valve partially deployed using the delivery device 1500, according to some embodiments; and

FIGS. 10 and 11 show variations on the prosthetic valve including modified frame configurations for the anchor frame subcomponent, according to some embodiments.

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### DETAILED DESCRIPTION

This disclosure is not meant to be read in a restrictive manner. For example, the terminology used in the application should be read broadly in the context of the meaning those in the field would attribute such terminology.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

The term “contiguous” refers to elements that share a common border or are touching.

The term “elastomer” refers to a polymer or a mixture of polymers that has the ability to be stretched to at least 1.3 times its original length and to retract rapidly to approximately its original length when released.

The term “elastomeric material” refers to a polymer or a mixture of polymers that displays stretch and recovery properties similar to an elastomer, although not necessarily to the same degree of stretch and/or recovery.

The term “film” includes coated and uncoated, filled and unfilled membranes, fabrics and the like.

The term “leaflet” as used in the context of prosthetic valves is generally a flexible component operable to move between an open and closed position under the influence of pressure differentials. For example, in operation, the leaflets open when an inflow fluid pressure exceeds an outflow fluid pressure and close when the outflow fluid pressure exceeds the inflow fluid pressure. In a closed position, the leaflet, alone or in combination with one or more other leaflets, operates to substantially restrict or obstruct (or alternatively completely obstruct) retrograde flowthrough the prosthetic valve. Thus, it will be appreciated that, in some instances, coaptation of adjacent leaflets may operate to completely block the flow of fluid (e.g., blood) through the prosthetic valve, while in other instances coaptation of adjacent leaflets may operate to block less than all of the flow of fluid (e.g., blood) through the prosthetic valve. In some embodiments, the leaflets include a free edge, and the free edges of adjacently situated leaflets coapt under the influence of outflow fluid pressure, thereby closing the valve so as to restrict or obstruct fluid from flowing retrograde through the prosthetic valve.

The term “non-elastomeric material” refers to a polymer or a mixture of polymers that displays stretch and recovery properties not similar to either an elastomer or elastomeric material, that is, considered not an elastomer or elastomeric material.

The term “prosthetic valve orifice” refers to a location into which a prosthetic valve may be placed. A prosthetic valve orifice includes a tissue orifice which includes anatomical structures into which a prosthetic valve can be placed. Such anatomical structures include, but are not limited to, a location wherein a cardiac valve may or may not have been surgically removed. Other anatomical structures that can receive a prosthetic valve include, but are not limited to, veins, arteries, ducts and shunts. A prosthetic valve orifice may also refer to a location in a synthetic or biological conduit that may receive a prosthetic valve.

The term “resilient” refers to the ability to recoil or spring back into shape after bending, stretching, or being compressed.

The term “tissue annulus” is inclusive of native cardiac valve structures, vasculature, and other anatomical features.

The term “transverse deformation resistance” as used herein refers to resistance to deformation in a substantially transverse plane to a longitudinal axis of a support structure. Examples of measures of transverse compressive resistance include radial compressive resistance, hoop strength, and flat plate stiffness, for example.

The term “tubular” as used herein includes tubes having a constant diameter along the length of the tube, and tubes having a variable diameter along the length of the tube, such as, but not limited to, a taper, anon-circular transverse profile or irregular circumference, and the like. For example, a tubular member may have a variable diameter along its length in at least one configuration of the tubular member. As another example, a tubular member may have a generally constant diameter in a delivery configuration, and a variable diameter in a deployed or pre-deployed configuration (e.g., when operably positioned in an anatomy of a patient).

The section headers in the description below are not meant to be read in a limiting sense, nor are they meant to segregate the collective disclosure presented below. The disclosure should be read as a whole. The headings are simply provided to assist with review, and do not imply that discussion outside of a particular heading is inapplicable to the portion of the disclosure falling under that header.

Although various examples are described herein in association with transcatheter designs, it is appreciated that the various examples of the prosthetic valve may be suitable for either surgical or transcatheter applications. Therefore, the inventive concepts described in association with transcatheter designs are applicable for both surgical and transcatheter applications and not limited to only transcatheter applications.

### Description of Various Embodiments

Inventive aspects of the examples addressed herein relate to configurations for achieving enhanced transverse deformation resistance in prosthetic valves utilizing overlapping, multi-frame subcomponent configurations, including associated systems and methods of use and manufacture thereof. Some examples relate to prosthetic valves used for cardiac valve replacement or other applications associated with native valve or other valve orifices, and related systems, methods, and apparatuses.

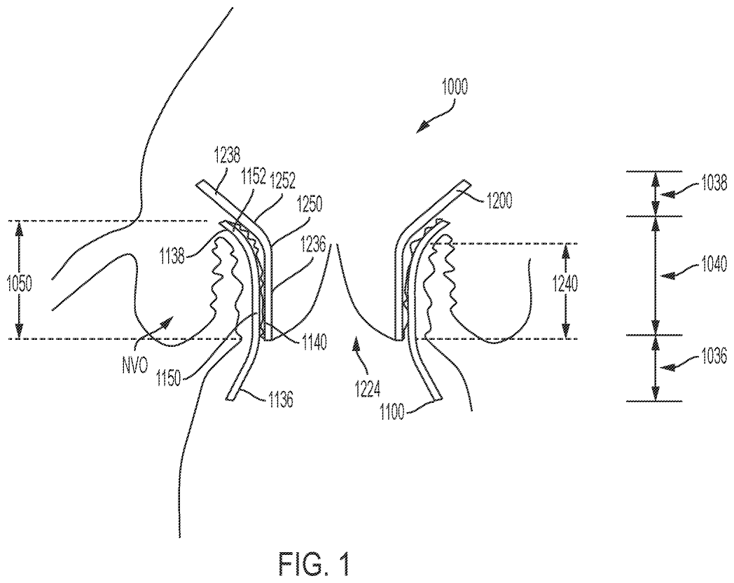

FIG. 1 is a schematic, sectional view a prosthetic valve 1000 implanted at a treatment site (e.g., in a native valve orifice NVO), according to some embodiments. In the instant disclosure, the examples are primarily described in association with surgical or transcatheter cardiac valve applications, although it should be readily appreciated embodiments within the scope of this disclosure can be applied toward any prosthetic valve or mechanism of similar structure and/or function. For example, the prosthetic valve 1000 of FIG. 1 can be applied in non-cardiac applications, such as respiratory or gastrointestinal tract applications.

As shown, the prosthetic valve 1000 has a multi-frame subcomponent configuration, where there are multiple, discrete frame subcomponents associated with the design. As shown, the prosthetic valve 1000 includes an anchor frame subcomponent 1100, a leaflet frame subcomponent 1200, and, optionally, an interstage subcomponent 1300 coupling the anchor frame subcomponent 1100 and the leaflet frame subcomponent 1200.

In various embodiments, the prosthetic valve 1000 is operable as a one-way prosthetic valve that defines a valve orifice into which leaflets open to permit flow and close so as to block or occlude the valve orifice and partially or entirely prevent flow in response to differential fluid pressure. The prosthetic valve 1000 is transitionable between a delivery configuration and a deployed configuration in-situ. In general terms, the leaflet frame subcomponent 1200 further operatively supports a leaflet assembly 1224 that functions as a one-way valve and the anchor frame subcomponent 1100 is operable to couple to an implant site (e g., the native valve orifice NVO, which may be that associated with an aortic valve). Where included, the interstage subcomponent 1300 is operable to permit the translation of the leaflet frame subcomponent 1200 into the anchor frame subcomponent 1100 during deployment. Further, in accordance with some embodiments, the interstage subcomponent 1300 is operable to permit perfusion during deployment of the prosthetic valve 1000.

The anchor frame subcomponent 1100 and the leaflet frame subcomponent 1200 are adapted to be transitioned from an un-nested configuration in which the leaflet frame subcomponent 1200 and the anchor frame subcomponent 1100 are separated from one another to a nested configuration in which the anchor frame subcomponent 1100 and the leaflet frame subcomponent 1200 engage along a combined interface corresponding to the interface region of the anchor frame subcomponent 1100 and the interface region of the leaflet frame subcomponent 1200 via an interference fit. In some examples, the anchor and leaflet frame subcomponents 1100, 1200 have complementary mating features, such as complementary shoulders to assist with component registration and/or anti-migration properties.

The combined interface of the multi-frame subcomponent arrangement presents a transverse deformation resistance that is enhanced, or greater than that of the leaflet and anchor frame subcomponents 1100, 1200 individually. In some examples, the interface regions of one or both of the anchor and leaflet frame subcomponents 1100, 1200 extends less than the total lengths of those subcomponents. In other examples, the interface regions extend the entire lengths of the anchor and leaflet frame subcomponents 1100, 1200. Regardless, it can be particularly beneficial that the combined interface corresponds to the portion of the leaflet frame subcomponent 1200 supporting the leaflet assembly of the prosthetic valve 1000 (e.g., along the coupling region from the leaflet base along the leaflet sides and commissures to the leaflet free edge). In particular, enhanced transverse deformation resistances helps maintain consistent support to the leaflet assembly by providing a consistent, pre-determined orifice, or inner lumen shape in which the leaflets can operate.

### Anchor Frame Subcomponent

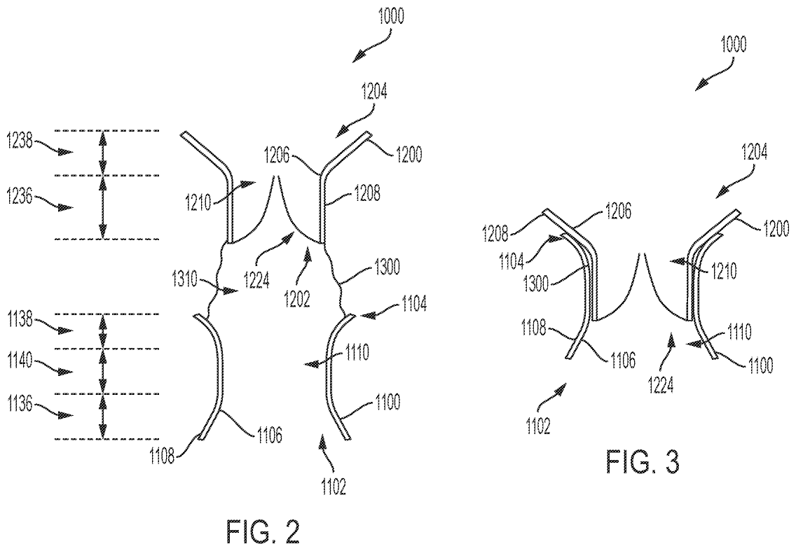

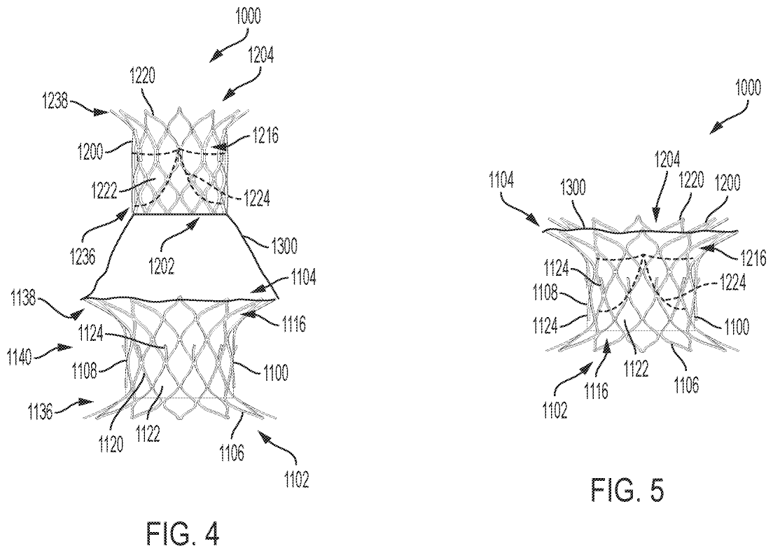

FIG. 2 is a sectional, schematic view of the prosthetic valve 1000 in an un-nested state, or configuration and FIG. 4 is a representation of the prosthetic valve 1000 in an un-nested configuration that shows additional, optional features (e.g., frame configurations). FIGS. 3 and 5 are similar representations, respectively, but in a nested configuration. As shown, the anchor frame subcomponent 1100 has an anchor frame inflow end 1102, also described as an inflow end 1102, an anchor frame outflow end 1104, also described as an outflow end 1104, an inner surface 1106, and an outer surface 1108, the inner surface 1106 defining an inner lumen 1110. The anchor frame subcomponent 1100 has a total length between the inflow end 1102 and outflow end 1104 and an inner diameter defined by the inner surface 1106. The inner lumen 1110 is a generally cylindrical void defined between the inflow end 1102 and the outflow end 1104, and the inner surface 1106. Though the inner lumen has a generally circular transverse cross-section along its length, in-situ, one or more portions of the inner lumen 1110 may adopt an irregular cross section, depending on the geometry of the tissue orifice into which it is placed and the conformity of the anchor frame subcomponent 1100 to the tissue annulus at the implant site. In various examples, the anchor frame subcomponent 1100 is configured to couple or otherwise be secured to a native valve orifice. Accordingly, in various examples, a diameter of the anchor frame subcomponent 1100 (e.g., a diameter of the outer surface 1108 is sized in accordance with patient anatomy).

It will be appreciated that nonlimiting examples of an anchor frame subcomponent 1100 can be provided with a diameter (e.g., a diameter of an exterior surface of the anchor frame subcomponent 1100) in a range of between twenty (20) millimeters and fifty (50) millimeters, depending on a patient's anatomy. However, diameters of more than fifty (50) millimeters are also envisioned, depending on patient anatomy. In general terms, in the deployed configuration the inner surface 1106 has a diameter or is otherwise sized similar to (e.g., the same or less than) one or more portions of the outer surface of the leaflet frame subcomponent 1200 to facilitate engagement of the leaflet frame subcomponent 1200 with the anchor frame subcomponent 1100 upon telescopically nesting the leaflet frame subcomponent 1200 in the anchor frame subcomponent 1100. Such telescopic nesting and engagement may be as a result of the “as manufactured” diameter of the anchor frame subcomponent 1100, or as a result of the “as expected” or “actual” diameter of the anchor frame subcomponent 1100 as deployed in the patient's anatomy.

In some embodiments, the anchor frame subcomponent 1100 includes an anchor frame 1120 and an anchor frame cover 1122 (e.g., as indicated on FIG. 4). The anchor frame 1120 may be at least partially covered by the anchor frame cover 1122 (e.g., a film or fabric) that is suitable for desired effect, such as to restrict fluid from passing through the wall of the anchor frame 1120, to encourage tissue ingrowth of the anchor frame subcomponent 1100 with the implant site, or alternative or additional purposes as desired. The anchor frame cover 1122 may be coupled to the inner surface, outer surface, or both inner surface and outer surface of the anchor frame 1120.

In various examples, the anchor frame subcomponent includes one or more tissue engagement features 1124, such as barbs or tissue anchors. In various examples, the one or more tissue engagement features 1124 project away from the anchor frame subcomponent 1100, radially outward from a longitudinal axis of the anchor frame subcomponent 1100, and toward the tissue surrounding the prosthetic valve 1000. If desired, the tissue engagement features 1124 may be oriented in an inflow direction (e.g., projecting outwardly and angled in an inflow direction) or an outflow direction (e.g., projecting outwardly and angled in an outflow direction). Generally, the tissue engagement features 1124 are operable to project away from the anchor frame subcomponent 1100 when the anchor frame subcomponent 1100 is deployed.

As shown in FIGS. 2 and 4, the anchor frame subcomponent 1100 has an inflow portion 1136, an outflow portion 1138, and an intermediate portion 1140 between the inflow and outflow portions 1136, 1138. The inflow portion 1136 defines an inflow flange or flare at the inflow end 1102 that flares or tapers radially outward when in the deployed configuration. The outflow portion 1138 defines an outflow flange or flared portion that flares or tapers radially outward when in the deployed configuration. As shown, each of the inflow and outflow portions 1136, 1138 have enlarged deployed diameters relative to the intermediate portion 1140. In various examples, such a configuration can help to minimize migration risks by facilitating abutment of the anchor frame subcomponent 1100 with native tissue annulus at the implant site. Moreover, the outflow portion 1138 may be configured to act as a shoulder feature to which the leaflet frame subcomponent 1200 can be engaged (e.g., to assist with proper positioning and/or avoiding unwanted migration of the leaflet frame subcomponent 1200 relative to the anchor frame subcomponent 1100).

Although the inflow and outflow portions 1136, 1138 can be integral with the intermediate portion 1140 as shown, it is also contemplated that separate, but attached portions may also be incorporated as desired. In-situ, either of the inflow or outflow portions 1136, 1138 may adopt irregular cross sections, depending on the geometry of the tissue orifice into which it is placed and the conformity of the particular portion. As shown, one or both of the inflow and outflow portions 1136, 1138 can flare outward in a trumpet shape having a concave curvature. In other embodiments, one or both of the inflow and outflow portions 1136, 1138 defines a convex curvature. Alternatively, a more linear, or non-curvilinear flare may be exhibited.

In various examples, the anchor frame 1120 of the anchor frame subcomponent 1100 is plastically deformable so as to be expandable via an expansion force (e.g., balloon expandable) or is self-expanding (e.g., by elastically recovering from a compacted profile to its deployed profile). For example, the anchor frame 1120 can comprise a shape memory material operable to flex under load and retain its original shape when the load is removed, thus allowing the anchor frame 1120, and the anchor frame subcomponent 1100 in general, to self-expand from a compressed shape to a predetermined larger shape. The anchor frame 1120 may comprise the same or different materials as the leaflet frame, described in further detail below. As referenced, in some examples, the anchor frame 1120 is configured to be plastically deformable such that it may be mechanically expanded from a compacted, delivery profile to an expanded, deployed profile by a radial expansion force, such as with a balloon. In yet some other examples, the anchor frame 1120 is plastically deformable as well as elastically deformable. That is, in some examples, the anchor frame 1120 includes one or more elastically deformable components or features and one or more plastically deformable components or features. Thus, it should be appreciated that the examples of the anchor frame 1120 presented herein are not to be limited to a specific design or mode of expansion.

In some examples, the anchor frame 1120 is capable of being expanded and locked into the expanded shape (e.g., by plastic deformation, expansion locking mechanism, or both). For example, in some instances the anchor frame 1120 includes a frame that, upon transitioning to a deployed configuration, includes a section with increased stiffness, or resistance to deformation in a transverse plane to a longitudinal axis of a device, including resistance to a change in shape, size, or both. Such an increase in transverse deformation resistance may be measured as an increase in radial compressive resistance or an increase in flat plate stiffness, for example, or both. Some examples of suitable frame designs with enhanced deformation resistance, and frame locking mechanisms, may be found in PCT Application No. PCT/US 2019/037998, filed Jun. 19, 2019.

In some embodiments, the anchor frame 1120 defines a tubular mesh having a framework defining apertures or voids 1116 (e.g., as shown in FIGS. 4 and 5). For example, the anchor frame 1120 optionally includes a plurality of frame members that are interconnected and arranged in one or more patterns of open cells (e.g., diamond-frame, open-cell patterns). In some examples, these patterns repeat one or more times over multiple rows. It should be appreciated that while the anchor frame 1120 is shown to include apertures or voids having generally a diamond shape, the interconnected frame members may be arranged in any number of alternative patterns, such as a series of sinusoids or any geometric shape that facilitate desired circumferential compressibility and expandability.

The anchor frame 1120 may comprise or otherwise be formed from a cut tube, or any other element. In some examples, the anchor frame 1120 is etched, cut, laser cut, or stamped into a tube or a sheet of material that is formed into a tubular structure. Alternatively, an elongated material, such as a wire, bendable strip, or the like, is bent or braided or otherwise formed into a tubular structure.

In terms of materials, the anchor frame 1120 can comprise any metallic or polymeric biocompatible material. For example, the anchor frame 1120 can comprise a material, such as, but not limited to nitinol, cobalt-nickel alloy, stainless steel, or polypropylene, acetyl homopolymer, acetyl copolymer, ePTFE, other alloys or polymers, or any other biocompatible material having adequate physical and mechanical properties to function as described herein.

In various embodiments, the anchor frame subcomponent 1100 is configured to provide positive engagement with an implant site to firmly anchor the prosthetic valve 1000 to the site. Such positive engagement with the implant site may be facilitated by one or more of the following, but not limited thereto: expansion spring bias of the anchor frame 1120; transverse deformation resistance of the expanded anchor frame 1120, tissue engagement features, and the geometric shape, contour and/or texture of the anchor frame 1120.

The anchor frame cover 1122 may be operable to prevent the flow of fluid through the wall of the anchor frame 1120 along all, or one or more portions of the anchor frame 1120. In various examples, the anchor frame cover 1122 is translucent or transparent, and thus the elements of the anchor frame 1120 are shown through the anchor frame cover 1122. In addition to inhibiting or preventing flow, the anchor frame cover 1122 may also be operable to provide a favorable surface for tissue abutment at the tissue annulus, and further, may be operable to facilitate tissue ingrowth at desired locations which may be advantageous for fixation of the prosthetic valve 1000 to the tissue annulus, facilitate a favorable biological response of the blood (e.g., to prevent a thrombotic response), and/or facilitate sealing of the prosthetic valve 1000 with the tissue orifice to minimize para-valvular leakage.

All or a majority of the voids of the anchor frame 1120 are covered by the anchor frame cover 1122 so as to block flow through the anchor frame 1120 (e.g., the anchor frame cover 1122 is less permeable to blood, such as being blood impermeable under physiologic conditions), or is configured to become less permeable to blood over time (e.g., similarly to woven and/or polyester-based graft materials). Thus, in some implementations, the anchor frame cover 1122 is a low permeability or impermeable film, sheet or membrane material coupled to the outer surface of the anchor frame 1120. The anchor frame cover 1122 may comprise any suitable material known in the art. By way of example, the anchor frame cover 1122 may be a film or fabric material, among others.

The anchor frame cover 1122 may be a sheet-like material that is biologically compatible and configured to couple to the anchor frame 1120. In various examples, the biocompatible material is a film that is not of a biological source and that is sufficiently flexible and strong for the particular purpose, such as a biocompatible polymer. In an embodiment, the film comprises a biocompatible polymer (e.g., ePTFE). In some examples, the film is a composite of two or more materials. The film may comprise one or more of a membrane, composite material, or laminate.

In various examples, the construction of and materials used in the film are such that the anchor frame cover 1122 is less permeable to blood (e.g., blood impermeable under physiologic conditions). In various examples, the construction of and materials used in the film are such that the anchor frame cover 1122 promotes cellular ingrowth, adhesion, and/or attachment. That is, in various examples, the anchor frame cover 1122 is constructed in a manner that promotes the ingrowth of tissue into one or more portions of the anchor frame cover 1122. It will be appreciated that cellular ingrowth may further increase sealing of the prosthetic valve with the tissue orifice and helps minimize para-valvular leakage, that is, leakage between the prosthetic valve and the tissue into which it is coupled.

### Leaflet Frame Subcomponent

As shown, the leaflet frame subcomponent 1200 is a generally tubular member having a leaflet frame inflow end 1202, also described as an inflow end 1202, a leaflet frame outflow end 1204, also described as an outflow end 1204, an inner surface 1206, and an outer surface 1208, the inner surface 1206 defining an inner lumen 1210. The inner lumen 1210 is a generally cylindrical void defined between the inflow end 1202 and the outflow end 1204, and the inner surface 1206. For reference, the inner lumen 1210 is a generally cylindrical void defined between the inflow end 1202 and the outflow end 1204, and the inner surface 1206.

The leaflet frame subcomponent 1200 (with the leaflet assembly 1224) generally provides the prosthetic valve 1000 with the functionality of a one-way valve. It is appreciated that mechanical leaflet, biological leaflet, synthetic leaflet, and biological and synthetic leaflet valves may be employed with the leaflet frame subcomponent 1200. It is also appreciated that, for transcatheter applications, the leaflet frame subcomponent 1200 is required to have a smaller-diameter compressed configuration and a larger-diameter expanded configuration, and that the valve and associated leaflets must be able to accommodate that functionality.

As shown, the leaflet frame subcomponent 1200 has an inflow portion 1236 and an outflow portion 1238. The outflow portion 1238 defines an outflow flange or flared portion that flares or tapers radially outward when in the deployed configuration. As shown, the outflow portion 1238 has an enlarged deployed diameter relative to the inflow portion 1236. In various examples, such a configuration can help to minimize migration risks and/or facilitate deployment by facilitating abutment of the leaflet frame subcomponent 1200 with the anchor frame subcomponent 1100. In particular, the outflow portion 1238 may be configured to act as a complementary shoulder feature to which the shoulder feature of the anchor frame subcomponent 1100 can be engaged (e.g., to assist with proper positioning and/or avoiding unwanted migration of the leaflet frame subcomponent 1200 relative to the anchor frame subcomponent 1100).

The leaflet frame subcomponent 1200 includes a leaflet frame 1220 (FIG. 4), a leaflet frame cover 1222 (FIG. 4), and a leaflet assembly 1224 (shown in broken lines) including one or more leaflets. In various embodiments, the leaflet frame subcomponent 1200 is configured to help provide positive engagement with an implant site to firmly anchor leaflet the prosthetic valve 1000 to the site. Such positive engagement with the implant site may be facilitated by one or more of the following, but not limited thereto: expansion spring bias of the leaflet frame 1220; transverse deformation resistance of the expanded leaflet frame 1220, tissue engagement features, and the geometric shape, contour and/or texture of the leaflet frame 1220.

In general terms, the leaflet frame 1220 helps provide structural support for the leaflet assembly 1224. The leaflet frame 1220 is operable to have a smaller delivery configuration diameter and a larger deployed configuration diameter, facilitated by balloon expansion and/or self -expansion deployment means.

In various examples, the leaflet frame 1220 of the leaflet frame subcomponent 1200 is plastically deformable so as to be expandable via an expansion force (e.g., balloon expandable) or is self-expanding (e.g., by elastically recovering from a compacted profile to its deployed profile). For example, the leaflet frame 1220 can comprise a shape memory material operable to flex under load and retain its original shape when the load is removed, thus allowing the leaflet frame 1220, and the leaflet frame subcomponent 1200 in general, to self-expand from a compressed shape to a predetermined larger shape. The leaflet frame 1220 may comprise the same or different materials as the leaflet frame, described in further detail below. As referenced, in some examples, the leaflet frame 1220 is configured to be plastically deformable such that it may be mechanically expanded from a compacted, delivery profile to an expanded, deployed profile by a radial expansion force, such as with a balloon. In yet some other examples, the leaflet frame 1220 is plastically deformable as well as elastically deformable. That is, in some examples, the leaflet frame 1220 includes one or more elastically deformable components or features and one or more plastically deformable components or features. Thus, it should be appreciated that the examples of the leaflet frame 1220 presented herein are not to be limited to a specific design or mode of expansion.

In some examples, the anchor frame 1120 is capable of being expanded and locked into the expanded shape (e.g., by plastic deformation, expansion locking mechanism, or both). For example, in some instances the anchor frame 1120 includes a frame that, upon transitioning to a deployed configuration, includes a section with increased stiffness, or resistance to deformation in a transverse plane to a longitudinal axis of a device, including resistance to a change in shape, size, or both. Such an increase in transverse deformation resistance may be measured as an increase in radial compressive resistance or an increase in flat plate stiffness, for example, or both. Some examples of suitable frame designs with enhanced deformation resistance, and frame locking mechanisms, may be found in PCT Application No. PCT/US 2019/037998, filed Jun. 19, 2019.

In some embodiments, the leaflet frame 1220 defines a tubular mesh having a framework defining apertures or voids 1216 (e.g., as shown in FIGS. 4 and 5). For example, the leaflet frame 1220 optionally includes a plurality of frame members that are interconnected and arranged in one or more patterns of open cells (e.g., diamond-frame, open-cell patterns). In some examples, these patterns repeat one or more times over multiple rows. It should be appreciated that while the leaflet frame 1220 is shown to include apertures or voids having generally a diamond shape, the interconnected frame members may be arranged in any number of alternative patterns, such as a series of sinusoids or any geometric shape that facilitate desired circumferential compressibility and expandability.

The leaflet frame 1220 may comprise or otherwise be formed from a cut tube, or any other element. In some examples, the leaflet frame 1220 is etched, cut, laser cut, or stamped into a tube or a sheet of material that is formed into a tubular structure. Alternatively, an elongated material, such as a wire, bendable strip, or the like, is bent or braided or otherwise formed into a tubular structure.

In terms of materials, the leaflet frame 1220 can comprise any metallic or polymeric biocompatible material. For example, the leaflet frame 1220 can comprise a material, such as, but not limited to nitinol, cobalt-nickel alloy, stainless steel, or polypropylene, acetyl homopolymer, acetyl copolymer, ePTFE, other alloys or polymers, or any other biocompatible material having adequate physical and mechanical properties to function as described herein.

The leaflet frame cover 1222 may be operable to prevent the flow of fluid through the wall of the leaflet frame 1220 along all, or one or more portions of the leaflet frame 1220. In various examples, the leaflet frame cover 1222 is translucent or transparent, and thus the elements of the leaflet frame 1220 are shown through the leaflet frame cover 1222. In addition to inhibiting or preventing flow, the leaflet frame cover 1222 may also be operable to provide a favorable surface for tissue abutment at the tissue annulus, and further, may be operable to facilitate tissue ingrowth at desired locations which may be advantageous for fixation of the prosthetic valve 1000 to the tissue annulus, facilitate a favorable biological response of the blood (e.g., to prevent a thrombotic response), and/or facilitate sealing of the prosthetic valve 1000 with the tissue orifice to minimize para-valvular leakage.

In some examples, all or a majority of the voids of the leaflet frame 1220 are covered by the leaflet frame cover 1222 so as to block flow through the leaflet frame wall (e.g., the leaflet frame cover 1222 is less permeable to blood, such as being blood impermeable under physiologic conditions), or is configured to become less permeable to blood overtime (e.g., similarly to woven and/or polyester-based graft materials). Thus, in some implementations, the leaflet frame cover 1222 is a low permeability or impermeable film, sheet or membrane material coupled at the outer surface 1208. The leaflet frame cover 1222 may comprise any suitable material known in the art. By way of example, the leaflet frame cover 1222 may be a film or fabric material, among others. For example, the leaflet frame cover 1222 may be a sheet-like material that is biologically compatible and configured to couple to the leaflet frame 1220. In various examples, the biocompatible material is a film that is not of a biological source and that is sufficiently flexible and strong for the particular purpose, such as a biocompatible polymer. In an embodiment, the film comprises a biocompatible polymer (e.g., ePTFE). In some examples, the film is a composite of two or more materials. The film may comprise one or more of a membrane, composite material, or laminate.

In various examples, the construction of and materials used in the film are such that the leaflet frame cover 1222 is less permeable to blood (e.g., blood impermeable under physiologic conditions). In various examples, the construction of and materials used in the film are such that the leaflet frame cover 1222 promotes cellular ingrowth, adhesion, and/or attachment. That is, in various examples, the leaflet frame cover 1222 is constructed in a manner that promotes the ingrowth of tissue into one or more portions of the leaflet frame cover 1222. It will be appreciated that cellular ingrowth may further increase sealing of the prosthetic valve with the tissue orifice and helps minimize para-valvular leakage, that is, leakage between the prosthetic valve and the tissue into which it is coupled.

In various embodiments, the leaflet assembly 1224, including one or more leaflets, is coupled to the leaflet frame 1220 to provide a one-way valve structure. As referenced above, a variety of mechanical valve, biological leaflet, and synthetic leaflet designs may be employed as desired. Examples of suitable leaflet constructions and methods of attachment to leaflet frames are illustrated and described in U.S. patent application Ser. Nos. 13/833,650, 14/973,589, and 14/622,599, the contents of each of which are incorporated herein by reference. Further examples of suitable leaflet materials are presented below.

In general terms, the leaflet assembly 1224 is coupled to the leaflet frame 1220 such that the one or more leaflets of the leaflet assembly 1224 are operable to open to allow flow from the inflow end 1202 to pass through the outflow end 1204, also referred to as the forward flow direction, and are operable to close to restrict flow from flowing from the outflow end 1204 through the inflow end 1202, also referred to as the retrograde flow direction.

The leaflet assembly 1224 can be coupled to the inner surface 1206 of the leaflet frame subcomponent 1200 (e.g., the frame 1220 and or cover 1222), the outer surface of the leaflet frame subcomponent 1200 (e.g., the frame 1220 and/or cover 1222), or both as desired. Various means for operably coupling the leaflet assembly 1224 to the frame 1220 and/or cover 1222 (e.g., stitching, adhesives, fasteners, and the like) are contemplated.

As previously referenced, in various embodiments, the leaflet frame subcomponent 1200 is nestable within the anchor frame subcomponent 1100. FIGS. 3 and 5 show the prosthetic valve 1000 with the anchor and leaflet frame subcomponents 1100, 1200 in the nested, expanded configuration. In terms of the full assemblies, the anchor frame subcomponent 1100 and the leaflet frame subcomponent 1200 are sized and shaped in a manner that provides for the leaflet frame subcomponent 1200 being coaxially disposable or receivable, or otherwise telescopically nested, at least partially within the anchor frame subcomponent 1100. In different terms, the anchor frame subcomponent 1100 is configured such that a portion of (or alternatively all of) the leaflet frame subcomponent 1200 can be received by or otherwise positioned within a space defined by the anchor frame subcomponent 1100 (e.g., to define a pair of adjacent inflow and/or outflow end portions).

### Leaflet Assembly Materials

In various examples, the one or more leaflets of the leaflet assembly 1224 are formed of a biocompatible, synthetic material (e.g., including ePTFE and ePTFE composites, or other materials as desired). In other examples, the leaflet(s) are formed of a natural material, such as repurposed tissue, including bovine tissue, porcine tissue, or the like.

Some examples of suitable leaflet materials may be found in U.S. Pat. No. 8,961,599 to Bruchman et al. (“Durable High Strength Polymer Composite Suitable for Implant and Articles Produced Therefrom”); U.S. Pat. No. 8,945,212 to Bruchman et al. (“Durable Multi-Layer High Strength Polymer Composite Suitable for Implant and Articles Produced Therefrom”); U.S. 9,554,900 to Bruchman et al. (“Durable High Strength Polymer Composites Suitable for Implant and Articles Produced Therefrom”); and U.S. Pat. App. Pub. 2015/0224231 to Bruchman et al. (“Coherent Single Layer High Strength Synthetic Polymer Composites for Prosthetic Valves”).

In accordance with embodiments herein, the leaflet material comprises a composite material having at least one porous synthetic polymer membrane layer having a plurality of pores and/or spaces and an elastomer and/or an elastomeric material and/or a non-elastomeric material filling the pores and/or spaces of the at least one synthetic polymer membrane layer. In accordance with other examples, the leaflet assembly 1224 further comprises a layer of an elastomer and/or an elastomeric material and/or anon-elastomeric material on the composite material. In accordance with examples, the composite material comprises porous synthetic polymer membrane by weight in a range of about 10% to 90%.

An example of a porous synthetic polymer membrane includes expanded fluoropolymer membrane having a node and fibril structure defining the pores and/or spaces. In some examples, the expanded fluoropolymer membrane is expanded polytetrafluoroethylene (ePTFE) membrane. Another example of porous synthetic polymer membrane includes microporous polyethylene membrane.

Examples of an elastomer and/or an elastomeric material and/or a non-elastomeric material include, but are not limited to, copolymers of tetrafluoroethylene and perfluoromethyl vinyl ether (TFE/PMVE copolymer), (per)fluoroalkylvinylethers (PAVE), urethanes, silicones (organopolysiloxanes), copolymers of silicon-urethane, styrene/isobutylene copolymers, poly isobutylene, polyethylene-co-poly(vinyl acetate), polyester copolymers, nylon copolymers, fluorinated hydrocarbon polymers and copolymers or mixtures of each of the foregoing. In some examples, the TFE/PMVE copolymer is an elastomer comprising between 60 and 20 weight percent tetrafluoroethylene and respectively between 40 and 80 weight percent perfluoromethyl vinyl ether. In some examples, the TFE/PMVE copolymer is an elastomeric material comprising between 67 and 61 weight percent tetrafluoroethylene and respectively between 33 and 39 weight percent perfluoromethyl vinyl ether. In some examples, the TFE/PMVE copolymer is a non-elastomeric material comprising between 73 and 68 weight percent tetrafluoroethylene and respectively between 27 and 32 weight percent perfluoromethyl vinyl ether. The TFE and PMVE components of the TFE-PMVE copolymer are presented in wt %. For reference, the wt % of PMVE of about 40, 33-39, and 27-32 corresponds to a mol % of about 29, 23-28, and 18-22, respectively.

In some examples, the TFE-PMVE copolymer exhibits elastomer, elastomeric, and/or non-elastomeric properties.

In some examples, the composite material further comprises a layer or coating of TFE-PMVE copolymer comprising from about 73 to about 68 weight percent tetrafluoroethylene and respectively from about 27 to about 32 weight percent perfluoromethyl vinyl ether.

In some examples, the leaflet material includes an expanded polytetrafluoroethylene (ePTFE) membrane having been imbibed with TFE-PMVE copolymer comprising from about 60 to about 20 weight percent tetrafluoroethylene and respectively from about 40 to about 80 weight percent perfluoromethyl vinyl ether, the leaflet material further including a coating of TFE-PMVE copolymer comprising from about 73 to about 68 weight percent tetrafluoroethylene and respectively about 27 to about 32 weight percent perfluoromethyl vinyl ether on the blood-contacting surfaces.

As discussed above, the elastomer and/or an elastomeric material and/or a non-elastomeric material may be combined with the expanded fluoropolymer membrane such that the elastomer and/or the elastomeric material and/or the non-elastomeric material occupies substantially all of the void space or pores within the expanded fluoropolymer membrane.

Although some examples of suitable leaflet materials have been provided, the foregoing examples are not meant to be read in a limiting sense, and additional or alternative materials are contemplated.

In some examples, the leaflet frame cover 1222, the anchor frame cover 1122, and/or the interstage subcomponent 1300 may be formed or otherwise include any of the above leaflet materials, including any natural/modified tissue materials, synthetic materials, and combinations thereof. Additionally, any of a variety of bio-active agents may be implemented with any of the foregoing. For example, any one or more of (including portions thereof) the leaflet frame subcomponent 1200 (e.g., the leaflet frame cover 1232), anchor frame subcomponent (e.g., anchor frame cover 1122), interstage subcomponent 1300 (e.g., tubular material) may comprise a bio-active agent. Bio-active agents can be coated onto one or more of the foregoing features for controlled release and/or activation of the agents once the prosthetic valve 1000 is implanted. Such coating methods can include, but are not limited to, those described in U.S. Pat. No. 9,101,696 to Leontein et al., filed Jul. 2, 2015. Such bio-active agents can include, but are not limited to, vasodilator, anti-coagulant, anti-platelet, and/or anti-thrombogenic agents such as, but not limited to, heparin. Other bio-active agents can also include, but are not limited to agents such as anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, m itoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6a-m ethylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate)); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retinoids; cyclin/CDK inhibitors; HMG co-enzyme reductase inhibitors (statins); and protease inhibitors.

### Interstage Subcomponent

FIGS. 2 and 4 show the interstage subcomponent 1300 extending between the anchor frame subcomponent 1100 and the leaflet frame subcomponent 1200 with the leaflet frame un-nested, or offset from the anchor frame subcomponent 1100. In various examples, the interstage subcomponent 1300 includes a flexible tubular material (e.g., membrane material) coupled about its circumference to the leaflet frame subcomponent 1200 at the inflow end 1202 and to the anchor frame subcomponent 1100 at the outflow end 1104. The interstage subcomponent 1300 is an optional feature than operates to couple the leaflet frame subcomponent 1200 to the anchor frame subcomponent 1100 in the unnested configuration. The interstage subcomponent 1300 may be thin and flexible, and operable to fold (evert) and/or elastically contract as desired.
When the anchor frame subcomponent 1100 and the leaflet frame subcomponent 1200 are expanded, the interstage subcomponent 1300 optionally defines a tapered configuration extending between the leaflet frame subcomponent 1200 and the anchor frame subcomponent 1100. The interstage subcomponent 1300 can be configured to facilitate nesting of the leaflet frame subcomponent 1200 into the anchor frame subcomponent 1100 and/or to retain the leaflet frame subcomponent 1200 nested within the anchor frame subcomponent 1100. For example, the interstage subcomponent 1300 may include reinforcement elements (described below) that help retain the nested configuration, or engagement of the interstage subcomponent 1300 between the leaflet frame subcomponent and anchor frame subcomponent 1100 may assist with maintaining the nested arrangement via a frictional, interference fit.
When the prosthetic valve 1000 is in the deployed nested configuration, and the leaflet frame subcomponent 1200 translated into the anchor frame subcomponent 1100 in a nested position, the interstage subcomponent 1300 is everted and positioned therebetween (e.g., sandwiched between the anchor frame subcomponent 1100 and the leaflet frame subcomponent 1200 as shown in FIG. 1).

As shown in FIG. 2, the interstage subcomponent 1300 has an inflow end 1302, and outflow end 1304, an inner surface 1306, and an outer surface 1308 (where the inflow and outflow ends 1302, 1304 are defined with the anchor and leaflet frame subcomponents 1100, 1200 in the deployed, nested configuration). As shown, the interstage subcomponent 1300 is coupled to the anchor frame subcomponent 1100 near the outflow end 1104 and is coupled to the inflow end 1202 near the inflow end 1302.

The interstage subcomponent 1300 may include a thin-walled flexible tubular member that defines an interstage subcomponent lumen 1310 (FIG. 2) in fluid communication with the inner lumen 1110 of the anchor frame subcomponent 1100 and the inner lumen 1210 of the leaflet frame subcomponent 1200 when in the pre-deployed configuration. When the leaflet frame subcomponent 1200 is nested into the anchor frame subcomponent 1100 the interstage subcomponent 1300 is operable to fold and evert so as to lie between the leaflet frame subcomponent 1200 and the anchor frame subcomponent 1100. The interstage subcomponent 1300 may comprise any suitable material known in the art. By way of example, the interstage subcomponent 1300 may be a film, fabric, or membrane, among others, that is flexible and less permeable to blood (e.g., blood impermeable under physiologic conditions).

The interstage subcomponent 1300 can be disposed within and/or about the anchor frame subcomponent 1100 and the leaflet frame subcomponent 1200 as desired. For example, the interstage subcomponent 1300 can extend not only between but also over or within either or both of the anchor frame subcomponent 1100 and the leaflet frame subcomponent 1200. In some examples, the interstage subcomponent 1300 is contiguous with the leaflet frame cover 1232 and the anchor frame cover 1122. In particular, the interstage subcomponent 1300 can be a contiguous film with that of the anchor frame cover 1122 and/or the leaflet frame cover 1232 that at least extends between and operates to couple the anchor frame subcomponent 1100 and the leaflet frame subcomponent 1200 to one another. As shown, the interstage subcomponent 1300 is formed from a generally tubular material and at least partially covers one or more of the anchor frame subcomponent 1100 and the leaflet frame subcomponent 1200.

The interstage subcomponent 1300 can include any sheet-like material that is biologically compatible and configured to couple to the anchor frame subcomponent 1100 and the leaflet frame subcomponent 1200. In various examples, the biocompatible material is a film that is not of a biological source and that is sufficiently flexible and strong for the particular purpose, such as a biocompatible polymer. In an embodiment, the film comprises a biocompatible polymer (e.g., ePTFE). The film may comprise one or more of a membrane, composite material, or laminate. In various examples, the construction of and materials used in the film are such that the interstage subcomponent 1300 has low permeability to fluid flow (e.g., blood impermeable) under physiologic conditions.

In various examples, the interstage subcomponent 1300 is impervious to fluid flow and controls the flow of fluid only through the interstage subcomponent lumen 1310 particularly during deployment of the prosthetic valve 1000 into the tissue orifice and acts as a low-permeability or impermeable seal between the leaflet frame subcomponent 1200 and the anchor frame subcomponent 1100 when in the deployed nested configuration.

During deployment of the prosthetic valve 1000, with the anchor frame subcomponent 1100 deployed within the tissue annulus and the leaflet frame subcomponent 1200 mounted to a delivery device 1500 (FIG. 9), blood flow may be occluded during deployment, or the interstage subcomponent 1300 may include features for facilitating selective blood flow during deployment of the prosthetic valve 1000. In particular, in some examples, the interstage subcomponent 1300 is operable to allow antegrade fluid flow, (e.g., blood perfusion) through the interstage subcomponent 1300 during deployment of the prosthetic valve 1000 into the tissue annulus.

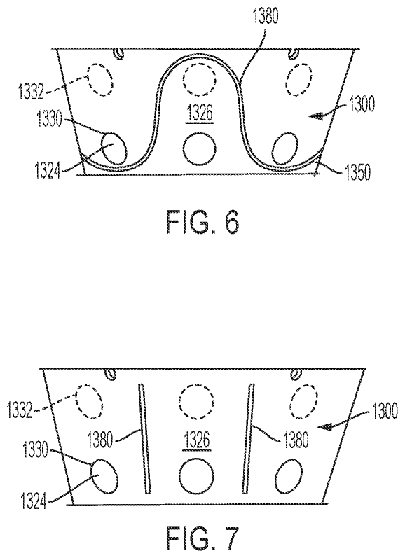

FIGS. 6 and 7 are views of different examples of the interstage subcomponent 1300 isolated from the prosthetic valve 1000. With reference to FIGS. 6 and 7, the prosthetic valve 1000 optionally includes one or more flow enabling features formed in the interstage subcomponent 1300.

In some embodiments, the interstage subcomponent 1300 comprises two layers of film, an inner film layer 1324 and an outer film layer 1326 with both layers coupled to either the inner or outer surface of the anchor frame 1120 and leaflet frame 1220, or the inner film layer 1324 bonded to the inner surfaces of the anchor frame 1120 and leaflet frame 1220 and the outer film layer 1326 coupled to the outer surfaces of the anchor frame 1120 and leaflet frame 1220.

In some examples, the inner film layer 1324 and the outer film layer 1326 are coupled together at least at the inflow end 1202 and the outflow end 1104. The inner film layer 1324 defines at least one inner film aperture 1332 therethrough adjacent the anchor frame subcomponent 1100 and the outer film layer 1326 defines at least one outer film aperture 1330 therethrough adjacent the leaflet frame subcomponent 1200.

The inner film layer 1324 and the outer film layer 1326 are not coupled at least between one of the inner film apertures 1332 and one of the outer film apertures 1330 so as to define a flow space therebetween such that the outer film layer 1326 lifts away from the inner film apertures 1332 to enable antegrade flow through the inner film apertures 1332 and the outer film apertures 1330 prior to the anchor frame subcomponent 1100 and the leaflet frame subcomponent 1200 being nested (while the anchor frame subcomponent 1100 and the leaflet frame subcomponent 1200 are longitudinally offset as illustrated and described herein). In some embodiments, the outer film layer 1326 is not coupled at least downstream of the outer film apertures 1330 and the inner film apertures 1332 so as to define the flow space.

In operation, the inner film layer 1324 and the outer film layer 1326 come together to close the flow space and to cover and seal the inner film apertures 1332 and outer film apertures 1330 under retrograde flow pressure and restrict or minimize retrograde flow through the inner film apertures 1332 and outer film apertures 1330. Further, the inner film layer 1324 and the outer film layer 1326 are configured to cover and seal the inner film apertures 1332 and outer film apertures 1330 when the leaflet frame subcomponent 1200 is nested into the anchor frame subcomponent 1100 and in a fully deployed configuration.

In the above embodiment, the inner film layer 1324 and the outer film layer 1326 are coupled together at least at the inflow end 1202 of the leaflet frame subcomponent 1200 and the outflow end 1104 of the anchor frame subcomponent 1100. It is appreciated that in accordance with an embodiment, the outer film layer 1326 may not be coupled together at or adjacent to the outflow end 1104 and still function to cover the inner film aperture 1332 during retrograde flow conditions.

Examples of such flow enabling features can also be found in U.S. Application Publication 2019/0110893, published Apr. 18, 2019, entitled “Telescoping Prosthetic Valve and Delivery System.”

In any of the examples of the interstage subcomponent 1300, the interstage subcomponent 1300 optionally includes one or more reinforcement elements 1380. In particular, FIG. 6 shows an optional reinforcement element 1380. The reinforcement element 1380 is optionally a stent-like frame element (e.g., a circumferentially-extending, sinuous shape memory element). FIG. 7 shows another example of reinforcement elements 1380 including one or more longitudinally extending reinforcement members (e.g., a fiber, wire, shape memory frame element or the like), or the like.

Additional examples of such reinforcement elements can be found in U.S. Application Publication 2019/0110893, published Apr. 18, 2019, entitled “Telescoping Prosthetic Valve and Delivery System.”

In various examples, the reinforcement element 1380 provides stiffening bias to the interstage subcomponent 1300, may be configured to evert along with the interstage subcomponent 1300, can be curved or s-shaped as shown or zig-zag, or take another form as desired. The one or more reinforcement elements 1380 can be temporarily elastically bent or folded upon itself as the anchor frame subcomponent 1100 and the leaflet frame subcomponent 1200 are nested to provide stiffening bias such that it takes a predetermined amount of force to nest the leaflet frame subcomponent 1200 into the anchor frame subcomponent 1100 and a corresponding predetermined amount of force to resist the movement of the leaflet frame subcomponent 1200 from the nested position. In some examples, with the anchor frame subcomponent 1100 and the leaflet frame subcomponent 1200 in the nested configuration, a column strength of the reinforcement element resists compressive loads that would otherwise cause the leaflet frame subcomponent 1200 to de-nest or telescope out of and away from the anchor frame subcomponent 1100. Although some functions and advantages of the one or more reinforcement elements 1380 have been described, additional or alternative features and advantages are contemplated.

Although various embodiments are described including the interstage subcomponent 1300, in other embodiments the interstage subcomponent 1300 is omitted. In such embodiments, the anchor frame subcomponent 1100 and leaflet frame subcomponent 1200 may not be coupled until delivery and deployment is completed.

### Multi-Frame Subcomponent Deployed Configuration

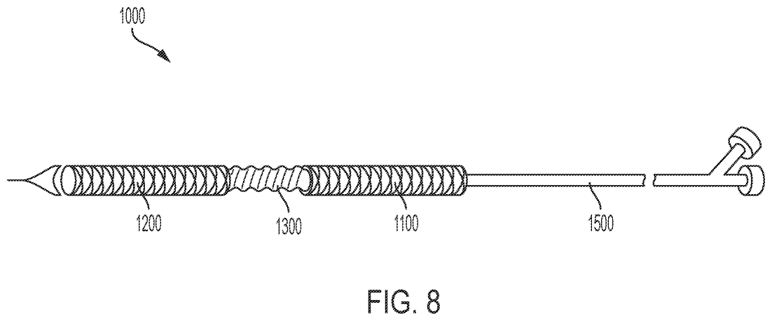

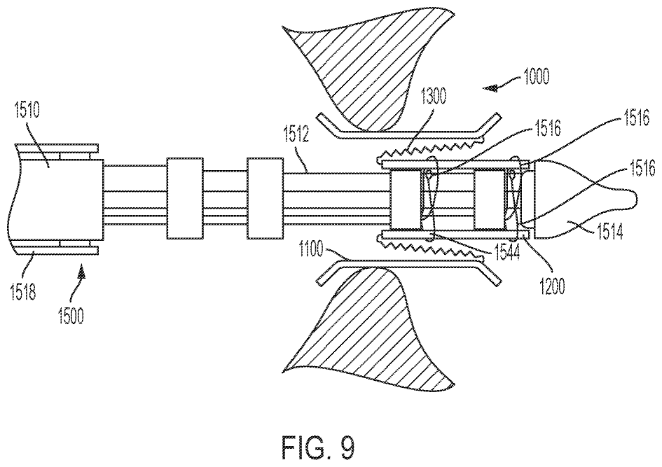

As previously noted, the prosthetic valve 1000 is transitionable between the delivery configuration (FIG. 8) and the deployed configuration (FIGS. 1, 3, and 5) in-situ. In some examples, the anchor frame subcomponent 1100 is configured to retain a compacted, undeployed profile until expanded to an expanded, deployed profile by an expansion force (e.g., the anchor frame subcomponent 1100 is configured to be balloon expandable). If desired, the leaflet frame subcomponent 1200 and/or the anchor frame subcomponent 1100 is configured to be self-expanding. For example, one or both may include a frame formed from shape memory material.

In the deployed configuration (FIG. 1) the anchor frame subcomponent 1100 anchors to tissue of a tissue annulus, such as the native valve orifice NVO, with the leaflet frame subcomponent 1200 partially or fully nested in the anchor frame subcomponent 1100. The anchor frame subcomponent 1100 has a first deployed transverse deformation resistance along the length of the anchor frame subcomponent 1100. The anchor frame subcomponent 1100 also defines, or includes an interface region 1150 that includes the intermediate portion 1140 and the inflow portion 1136 which defines a shoulder feature 1152. As shown, the interface region 1150 extends less than the total length of the anchor frame subcomponent 1100.

The leaflet frame subcomponent 1200, includes the leaflet assembly 1224 which is coupled to the leaflet frame subcomponent (e.g., leaflet frame and/or leaflet frame cover 1222) along a leaflet coupling region 1240. The leaflet frame subcomponent 1200 has a total length between the inflow end 1202 and an outflow end 1204 of the leaflet frame subcomponent 1200, an outer diameter. The leaflet frame subcomponent 1200 has a second deployed transverse deformation resistance along the length of the leaflet frame subcomponent 1200. The leaflet frame subcomponent 1200 also defines, or includes an interface region 1250 that includes the inflow portion 1236 and outflow portion 1238, and extends at least along the leaflet coupling region 1240. The interface region 1250 defines a complementary shoulder feature 1252 to the shoulder feature 1152 of the anchor frame subcomponent 1100.

In FIG. 1, the anchor frame subcomponent and the leaflet frame subcomponent have been transitioned from an un-nested configuration in which the leaflet frame subcomponent and the anchor frame subcomponent are separated from one another (e.g., FIGS. 2, 4, 8) to a nested configuration in which the anchor frame subcomponent 1100 and the leaflet frame subcomponent 1200 engage along a combined interface 1050 corresponding to the interface region 1150 of the anchor frame subcomponent 1100 and the interface region 1250 of the leaflet frame subcomponent 1200 via an interference fit. The combined interface 1050 optionally has a length that extends at least from an inflow side of the leaflet assembly to an outflow side of the leaflet assembly. The prosthetic valve 1000 has a combined interface transverse deformation resistance along the interface regions 1150, 1250 that exceeds each of the first and second deployed transverse deformation resistances of the anchor and leaflet frame subcomponents, 1100, 1200 respectively. In the nested configuration an outflow end of the prosthetic valve 1000 (e.g., corresponding to the outflow portion 1138 of the anchor frame subcomponent 1100) optionally has a transverse deformation resistance that is less than the combined interface transverse deformation resistance. In different terms, the overlapped regions of the two frame subcomponents optionally have greater transverse deformation resistance than the un-or non-overlapped regions.

If desired, by virtue of the overlapping arrangement, the prosthetic valve 1000 can includes an inflow region 1036, an outflow region 1038, and an intermediate region 1040 between the inflow and outflow ends of the prosthetic valve, where the intermediate region 1040 has the combined interface transverse deformation resistance and the inflow and outflow regions 1036, 1038 each have transverse deformation resistances that are less than the combined interface transverse deformation resistance. In the nested configuration the inner diameter of the anchor frame subcomponent and the outer diameter of the leaflet frame subcomponent are approximately the same along the leaflet coupling region 1240 of the leaflet frame subcomponent.

As shown, in the nested configuration, the prosthetic valve 1000 includes the inflow region 1036 defining a varying diameter to define an inflow taper corresponding to the inflow flange or flare at inflow end 1102 of the anchor frame subcomponent 1100. The outflow region 1038 also has a varying diameter to define an outflow tapered region corresponding to outflow flange or flare of the outflow portion 1238 of the leaflet frame subcomponent 1200. And, as shown, the intermediate region 1040 optionally has a relatively constant diameter to define an un-tapered intermediate region 1040. Such a consistent diameter may assist in promoting proper functioning of the leaflet assembly 1224, as previously described.

As shown, the shoulder feature and the complementary shoulder features 1152, 1252 include complementary tapered profiles. As shown, the shoulder feature 1152 and the complementary shoulder feature 1252 are optionally engaged and such that the shoulder features 1152, 1252 help with proper registration of the two frame subcomponents 1100, 1200 during nesting and/or help reduce or prevent migration of the leaflet frame subcomponent 1200 in a retrograde direction relative to the anchor frame subcomponent 1100. In different terms, the shoulder features 1152, 1252 have conformal shapes or interlock features that nest when pressed against one another to assist with registration and/or migration resistance.

As described, the interstage subcomponent 1300 can be implemented to couple the outflow end 1104 of the anchor frame subcomponent 1100 to the inflow end 1202 of the leaflet frame subcomponent 1200. Alternatively, the two components may be separate, and uncoupled prior to nesting. In the nested configuration, the interstage subcomponent 1300 is shown to define an everted configuration. If desired, the interstage subcomponent 1300 is operable to permit blood flow therethrough when the leaflet frame subcomponent 1200 is not nested in the anchor frame subcomponent 1100, and is operable to restrict flow therethrough when the leaflet frame subcomponent 1200 is nested within, and expanded to engage the anchor frame subcomponent 1100. If desired, the reinforcement elements 1380 of the interstage subcomponent 1300 are operable to assist with maintaining the leaflet frame subcomponent 1200 and the anchor frame subcomponent 1100 in the nested configuration. For example, those reinforcement elements 1380 may be hard to evert when the leaflet frame subcomponent 1200 is expanded and the anchor frame subcomponent 1100 is expanded, than when the leaflet frame subcomponent 1200 is unexpanded.

### Medical Device Including Prosthetic Valve and a Delivery Device

FIG. 8 shows the prosthetic valve 1000 mounted on a delivery device 1500, also described as a delivery device 1500, and FIG. 9 shows the prosthetic valve 1000 partially deployed using the delivery device 1500. As discussed above, in various examples, the prosthetic valve 1000 is loaded on a delivery device 1500 in a compacted, delivery configuration (also described as a pre-deployed configuration) with the anchor frame subcomponent 1100 and the leaflet frame subcomponent 1200 being longitudinally offset from one another (e.g., arranged in series).

A variety of delivery systems and associated deployment mechanisms (e.g., balloon expansion catheters, laparoscopic delivery systems, and others) are contemplated. In various examples, a fiber or constraint delivery system is employed where one or more constraining members releasably and independently couple the leaflet frame subcomponent 1200 and the anchor frame subcomponent 1100 to the delivery device 1500. In various examples, as discussed in greater detail below, the one or more constraining members can be selectively released from the leaflet frame subcomponent 1200 and the anchor frame subcomponent 1100 to facilitate in-situ nesting of the anchor frame subcomponent 1100 and the leaflet frame subcomponent 1200. In some examples, one or more of the constraining members include one or more portions that may be woven through the film(s) disposed about the leaflet frame subcomponent 1200 and the anchor frame subcomponent 1100, such that a longitudinal actuation of the delivery device 1500 is transferrable to one or more of the leaflet frame subcomponent 1200 and the anchor frame subcomponent 1100 via the one or more constraining members.

FIG. 9 is a side view of a delivery device 1500, according to some embodiments. As shown, the delivery device 1500 includes a body portion 1510, a support portion 1512, a tip portion 1514, a plurality of constraints 1516, and a constraining sheath 1518. In the view of FIG. 9, one or more constraints have been released from the anchor frame subassembly 1100 to permit expansion and deployment of the anchor frame subassembly 1100 in the valve orifice.

The plurality of constraints 1516 are adapted and arranged to interface with a respective one of the anchor frame subcomponent 1100 and the leaflet frame subcomponent 1200. The constraints 1516 generally include at least a first constraint and a second constraint for each of the anchor frame subcomponent 1100 and the leaflet frame subcomponent 1200 (the constraints 1516 for the anchor frame subcomponent 1100 are not shown in FIG. 9, as they have been released and retracted). In various embodiments, each of the plurality of constraints 1516 is formed as a fiber, strand, wire, combinations thereof or the like, and may be braided, wound, extruded, or otherwise formed of metallic or polymeric materials.

Examples of suitable delivery systems are described in Additional examples of such reinforcement elements can be found in U.S. Application Publication 2019/0110893, published Apr. 18, 2019, entitled ‘Telescoping Prosthetic Valve and Delivery System.“1000

### Method of Treating a Native Valve Orifice

As better understood with reference between FIGS. 9 and 1, a nonlimiting method of treating a native valve orifice (e.g., an aortic valve orifice) can be described. As shown, in FIG. 8, the prosthetic valve 1000 may be mounted to a delivery device 1500 with the leaflet frame subcomponent 1200 located distal, or longitudinally separate from the anchor frame subcomponent 1100 such that they are in an un-nested configuration. As shown in FIG. 9, the anchor frame subcomponent 1100 may be deployed first in a tissue annulus, such as the native valve orifice NVO, with a first pair of constraints (e.g., not shown) released such that the anchor frame subcomponent 1100 is operable to expand and engage the native valve orifice NVO (e.g., a valve annulus of an aortic valve, for example). However, as shown, first and second constraints 1544 and 1546 remain coupled about the leaflet frame subcomponent 1200.

With the anchor frame subcomponent 1100 unconstrained and the leaflet frame subcomponent 1200 at least partially constrained by the delivery device 1500, the leaflet frame subcomponent 1200 is withdrawn or retracted into the interior region defined by the anchor frame subcomponent 1100, as discussed herein. In various examples, the delivery device 1500 is withdrawn or retracted until the leaflet frame subcomponent 1200 becomes nested within the anchor frame subcomponent 1100, as discussed herein.

In some examples, before withdrawing or retracting the delivery device 1500 and the leaflet frame subcomponent 1200, a tension in one or more of the first and second constraints 1544 and 1546 may be reduced, thereby enabling the leaflet frame subcomponent 1200 to partially deploy. Thus, in such examples, the delivery device 1500 is operable to partially deploy the leaflet frame subcomponent 1200 prior to withdrawing the delivery device 1500 and the leaflet frame subcomponent 1200. Regardless, the leaflet frame subcomponent 1200 is expanded against the anchor frame subcomponent to define the combined interface 1050. Notably, although self-expanding anchor and leaflet frame subcomponents 1100, 1200 are described in this example, the retention features may be replaced or augmented with expansion means (e.g., one or more balloons) and the anchor and/or leaflet frame subcomponents 1100, 1200 may be configured to be radially expandable with an expansion force (e.g., being balloon expandable).

### Additional Information

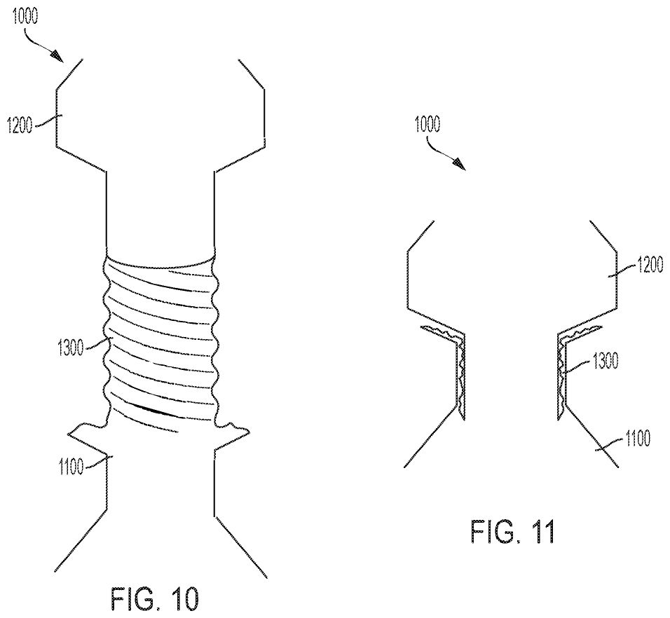

The scope of the concepts addressed in this disclosure has been described above both generically and with regard to specific examples. It will be apparent to those skilled in the art that various modifications and variations can be made in the examples without departing from the scope of the disclosure. For Example, FIGS. 10 and 11 show variations on the prosthetic valve 1000 including modified frame configurations for the anchor frame subcomponent 1100. In particular, as shown, the anchor frame subcomponent 1100 includes additional/alternative outflow tapered/flared portions. For example, the anchor frame subcomponent 1100 optionally includes additional primarily longitudinally-oriented portions (e.g., as in a right cylinder) and/or tapered (e.g., radially inwardly or outwardly) portions. From at least the foregoing, it should be clear that modifications are contemplated. And, likewise, the various components discussed in the examples discussed herein are combinable. Thus, it is intended that the examples cover the modifications and variations of the scope.

1. An apparatus, comprising a prosthetic valve comprising:
an anchor frame configured to anchor to tissue, including:
an anchor-frame inflow end; and
an anchor-frame outflow end;
a leaflet frame including:
a leaflet-frame inflow end; and
a leaflet-frame outflow end;
multiple prosthetic leaflets, mounted within the leaflet frame between the leaflet-frame inflow end and the leaflet-frame outflow end; and
an interstage tube coupling the anchor-frame outflow end to the leaflet-frame inflow end; wherein the prosthetic valve:
has an un-nested configuration in which the leaflet frame and the anchor frame are axially separated from one another by a gap, with the interstage tube bridging the gap;
has a nested configuration in which the leaflet frame is secured within the anchor frame; and
is configured to transition from the un-nested configuration to the nested configuration by the interstage tube everting and becoming sandwiched between the anchor frame and the leaflet frame to provide an interference fit between the anchor frame and the leaflet frame.

2. The apparatus according to claim 1, wherein:
the anchor frame has a first deployed transverse deformation resistance;
the leaflet frame has a second deployed transverse deformation resistance; and
in the nested configuration, a first interface region of the anchor frame and a second interface region of the leaflet frame combine to form a combined interface region having a combined interface transverse deformation resistance that exceeds each of the first and second deployed transverse deformation resistances.

3. The apparatus according to claim 2, wherein in the nested configuration the leaflet-frame outflow end and the anchor-frame inflow end have transverse deformation resistances that are less than the combined interface transverse deformation resistance.

4. The apparatus according to claim 1, wherein:
the anchor frame further includes a first interface region defining a shoulder feature;
the leaflet frame further includes a second interface region defining a complementary shoulder feature to the shoulder feature of the anchor frame; and
in the nested configuration, the first interface region and the second interface region combine to form a combined interface region with the shoulder feature and the complementary shoulder feature engaged.

5. The apparatus according to claim 4, wherein the shoulder feature and the complementary shoulder feature include complementary tapered profiles.

6. The apparatus according to claim 1, further comprising a tool configured to deliver the prosthetic valve to a heart of a subject, and to intracardially transition the prosthetic valve from the un-nested configuration to the nested configuration.

7. The apparatus according to claim 1, wherein:
the interstage tube includes an inner film layer and an outer film layer;
the inner film layer includes a first set of apertures; and
the outer film layer includes a second set of apertures that are non-overlapping with the first set of apertures.

8. The apparatus according to claim 1, wherein in the nested configuration, the interstage tube assumes defining an everted configuration.

9. The apparatus according to claim 1, wherein the interstage tube is operable to permit blood flow therethrough when the leaflet frame is not nested in the anchor frame, and is operable to restrict flow therethrough when the leaflet frame is nested within the anchor frame.

10. The apparatus according to claim 1, wherein the interstage tube comprises one or more reinforcement elements operable to maintain the leaflet frame and the anchor frame in the nested configuration.

11. The apparatus according to claim 1, wherein the interstage tube comprises a continuous sinuous reinforcement element formed separate and distinct from any frame element of the anchor frame and the leaflet frame.

12. The apparatus according to claim 1, wherein the prosthetic valve includes:
an inflow region having a varying diameter that defines an inflow taper;
an outflow region having a varying diameter that defines an outflow tapered region; and
an intermediate region between the inflow region and the outflow region, the intermediate region defining a constant diameter intermediate region.

13. The apparatus according to claim 1, wherein the anchor frame retains a compacted, undeployed profile until expanded to an expanded, deployed profile by an expansion force.

14. The apparatus according to claim 1, wherein the anchor frame is balloon expandable.

15. The apparatus according to claim 1, wherein at least one of the leaflet frame or the anchor frame is self-expanding.

16. The apparatus according to claim 1, wherein the leaflet frame is formed of shape memory material.

17. The apparatus according to claim 1, wherein the anchor frame includes a plurality of radially-projecting tissue anchoring barbs operable to engage tissue.

18. The apparatus according to claim 1, wherein:
the multiple prosthetic leaflets comprise a composite material including a matrix material and a filler material;
the matrix material includes a porous synthetic fluoropolymer membrane defining pores; and
the filler material includes a fluoropolymer material including a copolymer of tetrafluoroethylene and perfluoromethyl vinyl ether.

19. The apparatus according to claim 1, wherein each of the anchor frame and the leaflet frame include a cover configured to promote tissue ingrowth.

20. The apparatus according to claim 1, wherein the prosthetic valve includes a larger stiffness when in the nested configuration than when in the un-nested configuration.
